(19) United States
(12) Patent Application Publication
(10) Pub. No.: US 2004/0265825 A1
(43) Pub. Date: Dec. 30, 2004

(54) Methods and compositions for diagnosing and treating a subject having depression

(76) Inventor: Boris Tartakovsky, Rehovot (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/489,902

(22) PCT Filed: Jan. 17, 2002
(86) PCT No.: PCT/IL02/00046

Related U.S. Application Data
(60) Provisional application No. 60/262,780, filed on Jan. 19, 2001.
(60) Provisional application No. 60/306,507, filed on Jul. 18, 2001.

(51) Int. Cl. C12Q 1/68; G01N 33/574

(52) U.S. Cl. 435/6; 435/7.23

(57) ABSTRACT

This invention provides rapid sample processing, simultaneous analysis of individual cells, of defined cell populations which enables the measurements of apoptotic cellular marker protein level and function for the diagnosis of a subject with depression, monitoring of disease state and predicting and monitoring of therapeutic efficacy. This invention further provides anti-apoptotic compositions useful for the treatment of depression.

### FIELD OF THE INVENTION

The present invention relates to an assay for the determination of a state of depression in a subject. Further, this invention provides a method of diagnosing and monitoring a depressive disorder based on molecular, immunological or cytometric analysis of the level and/or function of one or more apoptotic cellular markers. In addition, this invention provides a method of diagnosing and predicting treatment's therapeutic outcomes and monitoring of treatment's therapeutic efficacy in subjects with mental disorders based on molecular, immunological or cytometric analysis of the level and/or function of one or more apoptotic cellular markers. Finally, this invention provides for new methods and compositions for treating depression.

### BACKGROUND OF THE INVENTION

Depression is a mental disorder characterized by Depressed mood, and markedly diminished interest or pleasure in activities. Other symptoms include significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts. A variety of somatic symptoms may also be present. Though depressive feelings are common, especially after experiencing setbacks in life, depressive disorder is diagnosed only when the symptoms reach a threshold and last at least two weeks. Depression can vary in severity from mild to very severe. It is most often episodic but can be recurrent or chronic. Some people have only a single episode, with a full return to premorbid function. However, more than 50 percent of those who initially suffer a single major depressive episode eventually develop another.

Depression is more common in women than in men. The point prevalence of unipolar depressive episodes is estimated to be 1.9% for men and 3.2% for women, and 5.8% of men and 9.5% of women will experience a depressive episode in a 12-month period. These prevalence figures vary across populations and may be higher in some populations.

Depression can affect individuals at any stage of the life span, although the incidence is highest in the middle ages. There is, however, an increasing recognition of depression during adolescence and young adulthood. Depression is essentially an episodic recurring disorder, each episode lasting usually from a few months to a few years, with a normal period in between. In about 20% of cases, however, depression follows a chronic course with no remission, especially when adequate treatment is not available. The recurrence rate for those who recover from the first episode is around 35% within 2 years and about 60% at 12 years. The recurrence rate is higher in those who are more than 45 years of age. One of the particularly tragic outcomes of a depressive disorder is suicide. Around 15% of depressive patients end their lives by committing suicide. Suicide remains one of the common and avoidable outcomes of depression.

Bipolar affective disorder refers to patients with a manic or a hypomanic episode with or without depressive episodes mania is characterized by elated mood, increased activity, over-confidence and impaired concentration. The point prevalence of bipolar disorder is around 0.4%. depressive episode within a bipolar disorder is designated bipolar depression in contrast to recurrent depressive episodes that are designated unipolar depression.

To summarize, depressive disorders are common mental disorders, causing a very high level of disease burden, and are expected to show a rising trend during the coming 20 years.

Although a genetic component has been suggested in depression, it has-not been confirmed, much less characterized. Further, it has not been reported whether the genetic bases and corresponding biochemical mechanisms underlying the different forms of depression are different in kind or only in degree. At present, no specific genetic or biochemical tests are available for the diagnosis of depression. Diagnosis and treatment monitoring are presently based clinical phenomenological criteria founded on clustering of signs and symptoms and the course of the disorder. As such diagnosis is based on subjective reports of patients and subjective elements in their evaluation and assessment by the clinician. The clinical heterogeneity associated with depression has complicated patients assessment, diagnosis and treatment. As a result, a large number of patients are underdiagnosed and under treated. Thus, as many as 50 percent of patients with depression in the community are not properly diagnosed or not effectively treated.

There is an urgent need for an objective biological assay for diagnosis and treatment monitoring of depression in a subject

### SUMMARY OF THE INVENTION

This invention provides a method for diagnosing a subject having depression comprising determining the mRNA or protein levels of depression-related genes, determining the percentage or level or amount of the cells in the subject which are apoptotic or the levels of markers of the apoptotic cells so as to determine the propensity of the cells to apoptosis, or determine the percentage of cells which are in late stage apoptosis whereby if the mRNA or protein levels of depression-related genes are increased or decreased compared to a non-depressed patient or if the percentage or level or amount of cells which are apoptotic or the markers of the apoptotic cells are greater or lower than a normative value the subject is diagnosed as having depression.

As provided herein, this invention provides a method for diagnosing a subject having depression by determining the percentage of cells in a sample from that subject that possess one or more markers. The markers may be a molecular marker, a cellular marker or a biochemical marker. The markers may be an apoptosis marker.

In the methods of the present invention, any apoptosis marker known to those of ordinary skill in the art will suffice. In one embodiment, the apoptotic cellular marker is phosphatidylserine. In another embodiment the marker is Annexin V. In another embodiment the apoptotic cellular marker is nuclear permeability.

In another embodiment, the marker is an apoptosis-related protein. The apoptosis-related proteins of the invention include but are not limited to those proteins listed in Table 1 described herein. These proteins include: Alpha-Catenin; Very Late Antigen; Apoptotic Protease-Activating Factor; Nucleoporin p62; Sma- and Mad-Related Proteins; Heat Shock Protein 60; Integrin 5 alpha protein; Tumor Necrosis Factor-1 Associated Death Domain; Extracellular Signal Regulated Kinases; Janus Kinase 1; Huntington-Associated Protein and Ceruloplasmin. Such proteins can be detected by procedures well known in the art such as electrophoresis, western blot antibody detection, ELISA (enzyme linked immunosorbent assay), chip technology including peptide chips, immunofluorescence including flow cytometry of fixed and permeabilized cells and the like. The antibodies utilized for detection may be monoclonal or polyclonal.

In another embodiment, the marker is depression-related gene expression. Gene expression may be monitored by procedures well known in the art such as northern analysis of RNA levels, PCR techniques, etc. In addition to the genes encoding the proteins in Table 1, mRNA levels for caspase 1, 5, 8, bak, Birc 3, Birc 6, Hus 1and Bcl2 may be monitored.

Gene expression may be increased or decreased during depression. The invention is related to an analysis of the differences in gene expression (increases/decreases) during depression.

The invention provides a method for diagnosing a subject having depression by determining the level of an apoptosis-related gene product. In one embodiment the apoptosis-related gene product is RNA. In another embodiment the apoptosis-related gene product is protein. In the methods of the invention, a blood sample is taken from a subject and the levels of one or more apoptosis-related gene products are determined.

In one embodiment, the invention is directed to a method of diagnosing a subject having depression a) obtaining a sample of cells of a subject and b) determining the level of one or more apoptotic related proteins in the cells thereby diagnosing the subject having depression.

In another embodiment, the invention is directed to a method of monitoring the progression of a depressive episode or depressive disorder of a subject by a) obtaining a sample of cells of a subject and b) determining the level of one or more apoptotic related proteins in the cells thereby monitoring the progression of a depressive episode or depressive disorder of the subject.

In another embodiment, the invention is directed to a method of monitoring the treatment of a depression disorder of a subject by a) obtaining a sample of cells of a subject and b) determining the level of one or more apoptotic related proteins in the cells thereby monitoring the treatment of the depression disorder in the subject.

In another embodiment, the invention is directed to a method of monitoring the progression of a depression disorder or treatment of a depression disorder of a subject by a) obtaining a sample of cells of the subject and b) determining the mRNA levels of one or more depression-related genes in the cells thereby monitoring the progression of a depression disorder of the subject.

In another embodiment, the invention is directed to a method of diagnosing a subject having depression by a) obtaining a sample of cells of the subject and b) determining the level of mRNA levels of one or more depression-related genes in the cells thereby diagnosing the subject having depression of the subject.

In another embodiment, the invention is directed to a method of monitoring the treatment of a depression disorder of a subject by a) obtaining a sample of cells of the subject and b) determining the mRNA levels of one or more depression-related genes in the cells thereby monitoring the treatment of the subject having depression.

In another embodiment, the method of diagnosing a subject having depression comprises the steps of a) obtaining a sample of cells of the subject; b) incubating the sample in serum-deprived media; c) contacting the sample with a binding molecule, wherein the binding molecule is capable of specifically binding to an apoptotic cellular marker, so as to form a complex between the binding molecule and the marker, d) determining the percentage of cells that possess the binding molecule in the sample, thereby diagnosing the subject having major depression. As provided herein, if the percentage of cells comprising the binding molecule or a label attached to the binding molecule is higher than a normative level the subject has depression.

This invention further provides methods of monitoring the progression of a depression disorder of a subject. In one embodiment the method comprises the steps of: a) obtaining a first sample of cells of the subject; b) incubating the sample in a serum-deprived media; c) contacting the sample with a binding molecule, wherein the binding molecule is capable of specifically binding to an apoptotic cellular marker, so as to form a complex between the binding molecule and the marker, d) determining the percentage of cells that possess the binding molecule in the first sample; e) obtaining a second sample of cells of the subject; f) incubating the sample in a serum-deprived media; g) contacting the second sample with a binding molecule, wherein the binding molecule is capable of specifically binding to an apoptotic cellular marker, so as to form a complex between the binding molecule and the marker; and h) determining the percentage of cells that possess the binding molecule in the second sample; comparing percentage of cells obtained in step d) with the level obtained the step h), thereby monitoring the progression of the subject having the depressive disorder.

This invention provides methods of monitoring the therapeutic efficacy of a treatment in a subject having a depressive disorder. In one embodiment, the method comprises the steps of: a) obtaining a first sample of cells of the subject; b) incubating the sample in a serum-deprived media; c) contacting the sample with a binding molecule, wherein the binding molecule is capable of specifically binding to an apoptotic cellular marker, so as to form a complex between the binding molecule and the marker; d) determining the percentage of cells that possess the binding molecule in the first sample; e) Treating the subject with an antidepressant, mood stabilizing or other relevant treatment or a combination of treatments; f) obtaining a second sample of cells of the subject; g) incubating the sample in serum-deprived media; h) contacting the second sample with a binding molecule, wherein the binding molecule is capable of specifically binding to an apoptotic cellular marker, so as to form a complex between the binding molecule and the marker; i) determining the percentage of cells that possess the binding molecule in the second sample; determining the therapeutic efficacy of the treatment based on the percentage of labeled cells, thereby monitoring the therapeutic efficacy of treatment in the subject having depression.

The invention further provides kits for diagnosing a subject having depression, for monitoring the progression of a depressive disorder in a subject and for monitoring the effect of treatment of a subject for depression. Such kits include all of the components necessary to determine the percentage or level or amount of cells in the subject that are apoptotic. Such components may include materials to detect apoptosis-related proteins such as those described in Table 1. Such materials may include electrophoresis, western blot antibody detection materials, chip materials, peptide chip, ELISA materials, immunofluorescence materials and the like. The kits of the invention may also include the necessary components to detect expression of apoptosis related genes. Such components may include materials for northern analysis and PCR analysis.

The invention further provides a method of determining the therapeutic effectiveness of a treatment for depression by determining the relative levels of one or more apoptosis-related gene products before and after treatment of a subject for depression.

This invention further relates to the use of apoptosis inhibitors for the treatment of depression and/or a depressive disorder. This invention is directed to methods of treating depression comprising administering one or more anti-apoptotic agents to a depressed patient. The invention is further directed to anti-depressant compositions comprising one or more anti-apoptotic agents. Such anti-apoptotic agents include drugs developed in order to block central nervous system (CNS) apoptosis in neurodegenerative diseases. Such drugs include, but are not limited to caspase inhibitors such as those available from Vertex and Idum Pharma; MP4 from Alexion Pharma, DP-B99 from D-Pharma and CoEnzyme Q10.

### BRIEF DESCRIPTION OF THE FIGURES

This invention will be better understood by reference to the Figures in which:

FIG. 1 shows the percentage of apoptotic lymphocytes (positive for Annexin V and negative for PI) derived from blood samples taken from healthy individuals and patients suffering from Depression and tested immediately after blood drawing (“time 0”).

FIG. 2 shows the percentage of apoptotic lymphocytes (positive for Annexin V and negative for PI) derived from blood samples taken from healthy individuals and patients suffering from Depression, that were cultured in vitro, overnight at 37 degrees centigrade and 5% C02, in RPMI medium supplemented with Fetal Calf Serum.

FIG. 3 shows the percentage of apoptotic lymphocytes (positive for Annexin V and negative for PI) derived from blood samples taken from healthy individuals and patients suffering from Depression, that were cultured in vitro, overnight at 37 degrees centigrade and 5% C02, in RPMI medium only (no Fetal Calf Serum).

FIG. 4 shows the forward scatter and side scatter characteristics of PBMC's after an overnight incubation (as in FIG. 3). R1 represents cells with a higher forward scatter, R2-cells with a smaller forward scatter and R3 represents cells from both R1 and R2. The results shown have been derived from the analysis of R3.

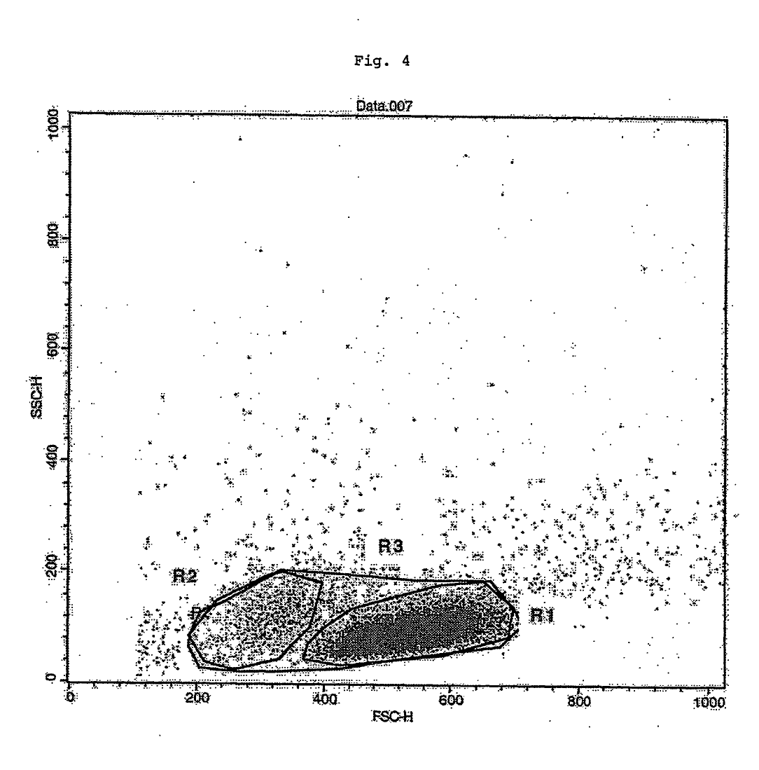

FIG. 5 shows the Annexin V (labeled with FITC, green fluorescence designated FL1) and PI (red fluorescence, designated FL9) staining of PBMC's after an overnight incubation. Cells positive for Annexin V and negative for PI (found in the lower, right quadrant) are apoptotic cells.

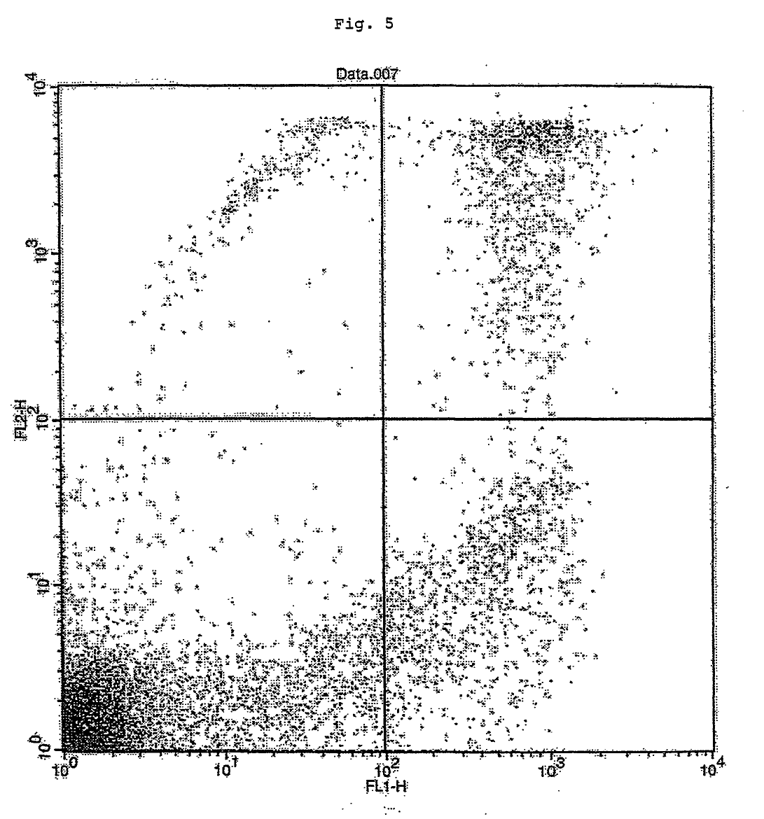

FIG. 6 shows the percent of apoptotic cells in 7 subjects suffering from Depression, before and after a successful drug treatment.

FIG. 7 shows the intracellular immunofluorescent staining of lymphocytes for the protein Tumor Necrosis Factor Receptor-1 Associated Death Domain protein (TRADD). Cells were obtained from healthy individuals and from patients suffering from Depression and tested immediately following blood drawing for the expression of TRADD.

### DETAILED DESCRIPTION OF THE INVENTION

The practice of the present invention will employ, unless otherwise indicated, conventional techniques of immunology, molecular biology, microbiology, cell biology and recombinant DNA, which are within the skill of the art. See, e.g., Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

In order to more fully understand the invention the following definitions are provided:

Definitions:

As used herein “inducing or increasing apoptosis” is defined as an increase in the rate of programmed cell death, i.e. more cells are induced into the death process.

As defined herein, “apoptotic cellular marker” means a cellular marker on the surface of the cell such as a surface protein or an intracellular marker such as a protein or RNA that is indicative of activation of the cell for apoptosis or that the cell is apoptotic. In one embodiment, the marker is plasma membrane changes such as phosphatidylserine appearance in the outer-membrane, cleavage of cytoskeletal proteins (fodrin and gelsolin) etc.; or nuclear changes such as chromatic condensation and specific degradation (nucleosomal ladder), cleavage of nuclear lamins; mitochondrial markers such as cytochrome c release, Bcl-xL, Smac/DIABLO release, mitochondrial membrane potential; cytosolic markers such as Bcl-2 family proteins, activated caspases, or NFₖB.

As defined herein, a “binding molecule” means a DNA, RNA, cDNA, protein, peptide, chimeric, ligand, oligomer, mimetic, or molecules which specifically binds to an apoptotic cellular marker.

“Incubating” means culturing the cells of a sample. The culturing may be in the presence or absence of serum. The incubation may also include culturing the sample in the presence of an apoptosis-stimulating agent or with a growth factor. Such incubation may be in the presence of serum or in a serum deprived condition.

“Serum deprived” means that the amount of serum is reduced compared to normal levels so that when cells isolated from depressed subjects are cultured in the serum deprived medium they exhibit a increase in apoptosis as compared to cells isolated from non-depressed subjects. Serum deprived includes but is not limited to serum free medium.

A “depression-related gene” is a gene whose expression is differentially regulated (increased or decreased expression) in patients diagnosed with a depressive disorder compared to expression in a patient lacking the depressive disorder.

A “depressive disorder” includes but is not limited to: major depressive disorder, single episode, recurrent major depressive disorder-unipolar depression, seasonal affective disorder-winter depression, bipolar mood disorder-bipolar depression, mood disorder due to a general medical condition-with major depressive-like episode, or mood disorder due to a general medical condition-with depressive features. Manic Depressive illnesses are also described in Goodwin, et al., 1990, Manic Depressive Illness, Oxford University Press, New York).

There are three types of depression generally characterized in the art, major depression, dysthymic disorder, or dysthymia, and depressive disorder not otherwise specified. Major depression is characterized by peak episodes of extreme depression. During a peak episode, the patient may suffer from depressed mood, and markedly diminished interest or pleasure in activities, . Other symptoms include significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts. Symptoms last for at least two weeks and cause significant distressor impairment in important areas of functioning.

Dysthymia is characterized by depressed mood for at least 2 years as well as other symptoms like poor appetite or overeating, insomnia or hypersomnia, low energy or fatigue, low self esteem, poor concentration or difficulty making decisions and feelings of hopelessness. As is recognized in the psychiatric art, depression may also comprise, and/or may also manifest itself in a variety of forms, including but not limited to, seasonal affective disorder, diurnal mood variations, or depression associated with menopause. Diagnostic criteria for dysthymia and major depression, as well as for seasonal affective disorder, diurnal mood variations and depression associated with menopause, are more fully explained in the Diagnostic and Statistical Manual of Mental Disorders, Fourth Edition, (DSM IV) published by the American Psychiatric Association or by the ICD (ICD-10: International Statistical Classification of Diseases and Related Health Problems (10th Revision) or any other psychiatric classification system.

Depression with seasonal affective pattern or seasonal affective disorder (hereinafter referred to as “SAD”) is also known as cabin fever, evening blues, and sun deprivation syndrome. The terms “seasonal affective disorder” or “seasonal pattern specifier” are defined in the DSM-IV as a specifier or adjective that more precisely characterize feature associated with depression. A particular feature of SAD is the regular occurrence of depression in winter.

Most of the patients with SAD are characterized by atypical type of depression in the winter which is associated with mood reactivity (mood brightens in response to acual or potential positive events) as well as weight gain or increase in appetite, hypersomnia, leaden paralysis (heavy, leaden feelings in arms or legs), long-standing pattern of interpersonal rejection sensitivity.

As defined herein, “visualizing” means that the complex of label and binding molecule of each of the apoptosis cellular markers may be visualized or detected by any means known in the art, including, but not limited to, ELISA, radioimmunoassay, peptide chip, flow cytometry, dot blots, Western immunoblotting combined with gel electrophoresis, immunohistochemistry, HPLC and mass spectrometry.

Specifically binds to an antibody” or “specifically immunoreactive with”, when referring to a protein or peptide, refers to a binding reaction which is determinative of the presence of the apoptotic cellular markers so as to determine the percentage of cells which are apoptotic in the presence of a heterogeneous population of proteins or cells. Thus, under defined immunoassay conditions, the specified antibodies bind to the apoptotic cellular marker antigens and do not bind in a significant amount to other antigens present in the sample. Specific binding to an antibody under such conditions may require an antibody that is selected for its specificity for a particular protein. For example, antibodies raised to the human apoptotic cellular marker immunogens described herein can be selected to obtain antibodies specifically immunoreactive with the apoptotic cellular markers proteins and not with other proteins. These antibodies recognize proteins homologous to the human apoptotic cellular markers protein.

A variety of immunoassay formats may be used to select antibodies specifically immunoreactive with a particular protein. For example, solid-phase ELISA immunoassays are routinely used to select monoclonal antibodies specifically immunoreactive with a protein. The antibodies may be detectably labeled, utilizing conventional labeling techniques well known to the art.

As used herein, the term “label” refers to a molecule, which may be conjugated or otherwise attached (i.e., covalently or non-covalently) to a binding molecule as defined herein. Particularly suitable labels include those, which permit analysis by ELISA, western blotting, and flow cytometry, e.g., fluorochromes. Preferred fluorochromes include phycoerythrin (P. E., Coulter Corp., Hialeah, Fla.), phycoerythrin-cyanin dye 5 (PECy5, Coulter), and fluorescein isothiocyanate (FITC, International Biological Supplies, Melbourne, Fla.). Other suitable detectable labels include those useful in colorimetric enzyme systems, e.g., horseradish peroxidase (HRP) and alkaline phosphatase (AP). Other proximal enzyme systems are known to those of skill in the art, including hexokinase in conjunction with glucose-6-phosphate dehydrogenase. Chemiluminescent labels, such as green fluorescent proteins, blue fluorescent proteins, and variants thereof are known. Also bioluminescence or chemiluminescence can be detected using, respectively, NAD oxidoreductase with luciferase and substrates NADH and FNIN or peroxidase with luminol and substrate peroxide. Other suitable label systems useful in the present invention include radioactive compounds or elements such as radioactive isotopes such as H³, I¹²⁵, I¹³¹ S³⁵ or immunoelectrodes.

The term “chip” refers to a a solid subsrate, for example silicon or glass having a surface to which one or more DNA, RNA or protein (peptide) templates are attached.

The term “protein chip” refers to chips for assaying proteins. Examples of protein chips include The Ciphergen ProteinChip® System available from Cipherphen which provides scientists with a versatile, integrated platform for biological research. Biologically important molecules from a variety of sources may be captured and analyzed on ProteinChip Arrays, using ProteinChip Readers and ProteinChip Software for rapid data analysis.

The term “polynucleotide”, “oligonucleotide”, or “nucleic acid” refers to a polymeric form of nucleotides of any length, either deoxyribonucleotides or ribonucleotides, or analogs thereof. The terms “polynucleotide” and “nucleotide” as used herein are used interchangeably. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A “fragment” or “segment” of a nucleic acid is a small piece of that nucleic acid.

A “gene” refers to a polynucleotide containing at least one open reading frame that is capable of encoding a particular protein after being transcribed and translated.

The terms “primer” and “nucleic acid primer” are used interchangeably herein. A “primer” refers to a short polynucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a polymerase and at a suitable temperature and pH. The primer may be either single-stranded or double-stranded and must be sufficiently long to prime the synthesis of the desired extension product. The exact length of the primer will depend upon many factors, including temperature, source of primer and use of the method.

A “polymerase chain reaction” (“PCR”) is a reaction in which replicate copies are made of a target polynucleotide using a “primer pair” or a “set of primers” consisting of an “forward” and a “reverse” primer, and a catalyst of polymerization, such as a DNA polymerase, and particularly a thermally stable polymerase enzyme. Methods for PCR are taught in U.S. Pat. No. 4,683,195 (Mullis) and U.S. Pat. No. 4,683,202 (Mullis et al.). All processes of producing replicate copies of the same polynucleotide, such as PCR or gene cloning, are collectively referred to herein as “amplificaton” or “replication”.

An apoptosis inhibitor is a compound, agent or molecule that inhibits or delays apoptosis or programmed cell death.

Taking into account these definitions, the present invention is directed to a method for diagnosing a subject having depression comprising determining the percentage or level or amount of the cells which are apoptotic or monitoring the levels of various apoptotic markers. Apoptosis or programmed cell death is the innate mechanism by which an organism eliminates unwanted cells. Cells undergoing apoptosis show a sequence of cardinal morphological features including membrane blebbing, cellular shrinkage and condensation of chromatin. Biochemically, these alterations are associated with the translocation of phosphatidylserine to the outer leaflet of the plasma membrane and the activation of an endonuclease that cleaves genomic DNA into multiples of internucleosomal fragments. In addition, various genes are expressed or repressed in apoptotic cells. In contrast, necrosis is classically induced following traumatic injury or exposure to high concentrations of noxious agents. Irreversible damage of the plasma membrane, mitochondrial dysfunction and cell lysis are characteristic for necrotic cell death.

Apoptosis

Cell death that occurs during normal tissue homeostasis was first reported to have unique histologic features by Kerr and colleagues. Kerr J F R, Willie A H, Currie A R. Apoptosis: A basic biological phenomenon with wide-ranging implications in tissue culture. J. Cancer 1972;26:239-257. These investigators noted that even in normal tissues, a small percentage of cells died each day and that this cell death could be recognized by its distinctive morphology and the failure to initiate an inflammatory response. This physiologic form of cell death was termed apoptosis. Subsequent studies have demonstrated that apoptosis represents a form of cellular suicide in which the dying cell initiates its own death through the activation of an internally encoded and evolutionarily conserved death program. Ellis R E, Yuan J, Horvitz H R. Mechanisms and functions of cell death. Annu Rev Cell Biol 1991 ;7:663-698, Chinnaiyan A M, Dixit V M. The cell-death machine. Curr Biol 1996;6:555-562 and Golstein P. Controlling cell death. Science 1997;275:1081-1082.

Apoptotic cell death can be triggered by a variety of extrinsic and intrinsic signals. Thompson C B. Apoptosis in the pathogenesis and treatment of disease. Science 1995;267:1456-1462. The physiologic control of apoptosis provides a mechanism for the elimination of cells that have been produced in excess, developed improperly, or sustained genetic damage. The hallmark of apoptosis is controlled auto digestion of the dying cell. Cell death appears to be carried out through the activation of endogenous proteases. Williams M S, Henkart P A. Apoptotic cell death induced by intracellular proteolysis. J Immunol 1994;153:42474255, Kumar S. ICE-like proteases in apoptosis. TIBS 1995;20:198-202. Deiss L P, Galinka H, Berissi H, Cohen O, Kimchi A. Cathepsin D protease mediates programmed cell death induced by interferon-g, Fas/APO-1 and TNF-α. EMBO J 1996;15:3861-3870, Vaux D L, Wilhelm S, Hncker G. Requirements for proteolysis during apoptosis. Mol Cell Biol 1997; 1 7:65026507

As a result of activation of these proteases, the integrity of the cytoskeleton is disrupted and the cell rounds up and begins to shrink in volume. In response to the contraction in cytoplasmic volume, the membrane begins to bleb and there is loss of the normal asymmetry of plasma membrane lipids. In healthy cells, phosphatidylserine is primarily distributed to the inner leaflet of the plasma membrane. During apoptosis, phosphatidylserine becomes exposed on the outer leaflet of the plasma membrane Castedo M, Hirsch T, Susin S A, et al. Sequential acquisition of mitochondrial and plasma membrane alterations during early lymphocyte apoptosis. J lmmunol 1996;157:512-521. Endonucleases are activated and begin to degrade nuclear DNA. In some cell types, DNA is degraded into fragments the size of oligonucleosomes, whereas in others larger DNA fragments are produced. Degraded DNA associated with death or apoptosis of cells can be detected by the binding of 7-amino actinomycin D (7AAD).

A key feature of apoptosis is that the plasma membrane remains intact. The alterations of the plasma membrane, including the exposure of phosphatidylserine, signal neighboring phagocytic cells to engulf the apoptotic cell and complete the degradation process. Phosphatidylserine exposure can be detected by the binding of Annexin V. Apoptosis also involves characteristic changes within the nucleus. By maintaining plasma membrane integrity, apoptotic death promotes the elimination of the dying cell without the induction of an inflammatory response.

Apoptosis Detection Techniques

Apoptosis can be measured by numerous procedures well know in the art. Specific examples of apoptosis assays are provided in the following references. These procedures include but are not limited to the propidium iodide flow cytometry assay described in Dengler et al., (1995) Anticancer Drugs. 6:522-32, or by the in situ terminal deoxynucleotidyl transferase and nick translation assay (TUNEL analysis) described in Gorczyca, (1993) Cancer Res 53:1945-51. Assays for apoptosis in lymphocytes are disclosed by: Li et al., Science 268:429-431, 1995; Gibellini et al., Br. J. Haematol. 89:24-33,1995; Martin et al., J. Immunol. 152:330-342,1994; Terai et al., J. Clin Invest. 87:1710-1715,1991; Dhein et al., Nature 373:438-441, 1995; Katsikis et al., J. Exp. Med. 1815:2029-2036,1995; Westendorp et al., Nature 375:497,1995; DeRossi et al., Virology 198:234-244, 1994. Assays for apoptosis in fibroblasts are disclosed by: Vossbeck et al., Int. J. Cancer 61:92-97,1995; Goruppi et al., Oncogene 9:1537-1544, 1994; Fernandez et al., Oncogene 9:2009-2017, 1994; Harrington et al., EMBO J., 13:3286-3295, 1994; Itoh et al., J. Biol. Chem. 268:10932-10937, 1993. Assays for apoptosis in neuronal cells are disclosed by: Melino et al., Mol. Cell Biol. 14:6584-6596,1994; Rosenbaum et al., Ann. Neurol. 36:864-870, 1994; Sato et al., J. Neurobiol 25:1227-1234, 1994; Ferrari et al., J. Neurosci. 1516:2857-2866, 1995; Talley et al., Mol. Cell Biol. 1585:2359-2366,1995; Talley et al., Mol. Cell. Biol. 15:2359-2366,1995; Walkinshaw et al., J. Clin. Invest. 95:2458-2464, 1995. Assays for apoptosis in insect cells are disclosed by: Clem et al., Science 254:1388-90, 1991; Crook et al., J. Virol. 67:2168-74, 1993; Rabizadeh et al., J. Neurochem. 61:2318-21, 1993; Birnbaum et al., J. Virol. 68:2521-8,1994; Clem et al., Mol. Cell. Biol. 14:5212-5222,1994.

Flow cytometry may be used to detect apoptosis. Real time analysis of the assembly of ligand, receptor, and apoptotic cellular markers by quantitative fluorescence flow cytometry is also provided. The following patents teach cytometric methods: U.S. Pat. Nos. 5,915,925, 5,895,764, 5,880,474, 5,858,667, 5,853,984, 5,840,478, 5,837,547, 5,808,737, 5,776,781, 5,776,754, 5,776,711, 5,763,201, 5,757,476, 5,757,475, 5,739,902, 5,736,330, 5,736, 5,731,867, 5,726,751, 5,726,364, 5,700,692, 5,690,895, 5,684,575, 5,675,517, 5,643,796, 5,641,457, 5,631,730, 5,627,040, 5,620,842, 5,605,805, 5,602,349, 5,602,039, 5,582,982, 5,542,305, 5,540,494, 5,504,337, 5,483,469, 5,478,722, 5,475,487, 5,466,572, 5,464,581, 5,451,525, 5,437,200, 5,434,081, 5,432,089, 5,412,466, 5,395,588, and 5,314,824.

Further, cells may be detected using standard flow cytometry analysis using FACscan or FACS Calibur analyzers (Becton Dickinson, San Jose, Calif.). Cytometric techniques are known to those skilled in the art. For example the following describe such techniques: U.S. Pat. No. 5,298,426 Method of differentiating erythroblasts from other cells by flow cytometry; U.S. Pat. No. 5,296,378 Method for classifying leukocytes by flow cytometry; U.S. Pat. No. 5,270,548 Phase-sensitive flow cytometer; U.S. Pat. No. 5,247,340 Flow imaging cytometer; U.S. Pat. No. 5,179,026 Method of classifying leukocytes by flow cytometry.

Reagents used in the cytometric method include: U.S. Pat. No. 5,175,109 Reagent for classifying leukocytes by flow cytometry; U.S. Pat. No. 5,167,926 Apparatus for pretreating cells for flow cytometry; U.S. Pat. No. 5,160,974. Closed sample cell for use in flow cytometry; U.S. Pat. No. 5,159,403 Flow cell mechanism in flow imaging cytometer; U.S. Pat. No. 5,159,398 Flow imaging cytometer; U.S. Pat. No. 5,150,313 Parallel pulse processing and data acquisition for high speed, low error flow cytometry; U.S. Pat. No. 5,144,224 Millimeter wave flow cytometer; U.S. Pat. No. 5,093,234 Method of aligning, compensating, and calibrating a flow cytometer for analysis of samples, and microbead standards kit therefor; U.S. Pat. No. 5,073,497 Microbead reference standard and method of adjusting a flow cytometer to obtain reproducible results using the microbeads U.S. Pat. No. 5,039,613 Reagents used in a method of classifying leukocytes cytometry U.S. Pat. No. 5,032,381 Chemiluminescence-based static and flow cytometry; and U.S. Pat. No. 4,954,715 Method and apparatus for an optimized multiparameter.

One can also use protein assasys such as immunoassays to detect protein apoptotic cellular markers. Either monoclonal or polyclonal antibodies (as well as any recombinant antibodies) specific for the apoptosis cellular markers can be used in various-immunoassays. Such assays include competitive immunoassays, protein chip assays, radioimmunoassays, Western blots, ELISA, indirect immunofluorescent assays and the like. The antibodies directed to the apoptotic cellular markers may be coupled to a solid-phase support, e.g., nitrocellulose, nylon, column packing materials (e.g., Sepharose beads), magnetic beads, on a chip, array, glass wool, plastic, metal, polymer gels, cells, or other substrates. Such supports may take the form, for example, of beads, wells, dipsticks, or membranes.

One can also use analysis of RNA levels to detect apoptotic and or depression-related cellular markers. DNA probes may be utilized to measure and quantitate RNA levels of apoptotic cellular markers. Such probes may be cloned DNA or synthetic oligonucleotides.

As contemplated herein, the measurement of the level and function of apoptotic cellular markers in cells may in one embodiment require cell permeabilization to allow the binding of the labeled binding molecule which is specific for apoptotic cellular markers, to the intracellular components. As used herein, a permeabilizing agent is any compound that facilitates access of a below-described detecting agent to the cytoplasm of the cell. Permeabilization is known to those skilled in the art. For example, permeabilization incubation with digitonin is described [Fiskurn et al., 1980, Proc. Natl. Acad Sci. USA, 77:3430-3434; Anderson et al., 1989, J Immunol., 143:1899-1904). The incubation may be performed on ice for between about 5 minutes to about 30 minutes. However, the skilled artisan can readily adjust these conditions, as needed or required. Although less desirable, other means of cell permeabilization may be utilized in the method of the invention. For example, the cells may be permeabilized by incubation with 0.05% to 0.1% paraformaldehyde prior to incubation with digitonin. The means of permeabilization are not a limitation on the present invention.

Any permeabilizing agent which provides cells that are intact and suitable for the purpose of the analysis is useful for the invention. Permeabilizing agents include but are not limited to those which unmask nucleic acids from associated proteins, form pores that allow access of the below described detecting agent to the cytoplasm, or that extract lipid from the outer cell membrane and allow access of the detecting agent to the underlying cytoplasm. Particularly preferred permeabilizing agents that unmask nucleic acid from protein include Proteinase K, pronase E, dispase, diastase, papain, trypsin and pepsin/HCl for animal cells; cellulase or pectinase for plant cells; and lysozyme for bacterial cells. Non-chemical means such as cycles of freezing followed by thawing of cells or microwave irradiation can also be used for permeabilizing. Permeabilizing agents that form pores that allow access of the detecting agent to the cytoplasm include detergents such as saponin, sodium dodecyl sulphate, CHAPS™, Triton-XI0O, Brij35′m and Brij5C. Permeabilizing agents that extract lipid from the outer cell membrane are known in the art and include, for example, alcohols such as ethanol or methanol which may be used in combination with other compounds including acids such as acetic acid, or acetone. Some fixatives such as formaldehyde and alcohol-based fixatives also act as permeabilization agents.

Diagnosing Depression

The assays of the present invention are used in conjunction with conventional methods of diagnosing depression. The diagnosis of depression usually follows a clinical evaluation by a psychiatrist or other mental health professionals. The two most recognized sets of diagnostic criteria for major depressive disorder and other depressive, or mood disorders, are outlined in the DSM, Diagnostic and Statistical Manual of Mental Disorders, Fourth Edition, (DSM IV) published by the American Psychiatric Association and the ICD (ICD-10: International Statistical Classification of Diseases and Related Health Problems-10th Revision, published periodically by the World Health Organization) or any other psychiatric classification system.

The presence and the severity of the depressive state can also be determined with structured and semi-structured interview and questioners such as the Hamilton score that is well known in the art. Hedlung, et al. The Hamilton Rating Scale for Depression, Journal of Operational Psychiatry (1979) 10 (2) 149-165. The molecular and biochemical assays are used to confirm the classical Hamilton score diagnoses.

Treating Depression

Once a patient is diagnosed with depression, there are various treatments modalities available to treat the depression. The treatments include, but are not limited to: antidepressants: biogenic amine non-selective reuptake inhibitors, e.g., tricyclic antidepressants like Imipramine; serotonin selective reuptake inhibitors like Fluoxetine (Prozac); monoamine oxidase inhibitors (MAO-In) like phenelezine; other types of antidepressant medications including atypical antidepressants. Antidepressants augmentation with other medications e.g., lithium, T3, T4, etc. Other treatment modalities with antidepressant effects: electro convulsive treatment (ECT); light therapy psychotherapy e.g., cognitive or interpersonal therapy for depression.

In addition, administration of compounds, in particular drugs, reported to ameliorate or exacerbate the symptoms of a neuromental disorder, include but are not limited to compounds include antidepressants such as lithium salts, carbamazepine, valproic acid, lysergic acid diethylamide (LSD), p-chlorophenylalanine, p-propyidopacetamide dithiocarbamate derivatives e.g., FLA 63; anti-anxiety drugs, e.g., diazepam; monoamine oxidase (MAO) inhibitors, e.g., iproniazid, clorgyline, pheneizine and isocarboxazid; biogenic.amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), S and dibenzodiazepines (e.g., clozapine); benzodiazepines; dopaminergic agonists and antagonists e.g., L-DOPA, cocaine, amphetamine, a-methyl-tyrosine, reserpine, tetrabenazine, benzotropine, pargyline; noradrenergic agonists and antagonists e.g., clonidine, phenoxybenzamine, phentolamine, tropolone. In another embodiment of the treatment methods, the compounds administered comprise compounds, in particular drugs, reported to ameliorate or exacerbate the symptoms of oxidative stress disorder. Such compounds include reduced IS glutathione (GSH), glutathione precursors, e.g., N-acetylcysteine; antioxidants, e.g., vitamins E and C, beta carotene and quinones; inhibitors of lipid membrane peroxidation, e.g., 21-aminosteroid U74006F (tirilazad mesylate), and lazaroids; antioxidants such as mazindol; 2c dizocilpine maleate; selegiline; sulfhydryls N-acetyleysteine and cysteamine; dimethylthiourea; EUK-8 a synthetic, low molecular salen-manganese complex; synthetic manganese-based metalloprotein superoxide dismutase mimic, SC52608; free radical scavengers or suppressors, e.g., pegorgotein, tocotrienol, tocopheral, MDL 74,18, LY231617, MCI-186, AVS (nicaraven), allopurinol, rifampicin, oxypurinol, hypochlorous acid or recombinant human Cu,Zn-SOD.

In addition to known methods of treating depression, the present invention is further directed to compositions of apoptosis inhibitors for treatment of depression and/or depressive disorders. The present invention is further directed to methods of treating depressive disorders or depression with apoptosis inhibitors. Such apoptosis inhibitors are well known in the art and include but are not limited to serotonin, dopamine, ascorbic acid, gluguidone, caffeine, hydrocortisone and dexamethasone as described in U.S. Pat. No. 5,840,719; polypeptides having the ART domain and the BAX domain as described in U.S. Pat. No. 6,245,885; methylsphingosine as described in U.S. Pat. No. 5,583,160; Fadd-like anti-apoptotic molecules such as flame-1 and flame-2 as described in U.S. Pat. No. 6,063,760; dipeptide apoptosis inhibitors such as those described in U.S. Pat. No. 6,184,210; cysteine or serine protease inhibitors coupled with photodynamic therapy as described in U.S. Pat. No. 6,180,402 and inhibitors such as those described in U.S. Pat. Nos. 6,228,603, 6,046,007 and 6,015,665.

Furthermore, the antidepressive compositions of the invention may include one or more drugs developed in order to block central nervous system (CNS) apoptosis in neurogenerative diseases including, for example, caspase inhibitors such as those available from Vertex and Idun Pharma, MP4 from Alexion Pharma, DP-B99 from D-Pharma and CoEnzyme Q10 from Receptogen. In addition, various anti-apoptosis agents that find use in the invention include those directed at the web site the infoshop.com/study/bc5995 new growth opportunities.

Monitoring Treatments for Depression

Once a subject has been treated for depression, he/she is monitored for depression symptoms by conventional analysis techniques as described above and using the assays described herein.

The invention having been described is now illustrated by the following non-limiting Examples.

### EXAMPLES

Experiments were performed on peripheral blood mononuclear cells (PMBC) derived from healthy and depressed individuals as described in the following examples. A psychiatrist diagnosed depressed patients using the diagnostic criteria outlines by the DSM or the ICD (Diagnostic and Statistical Manual of Mental Disorders, Fourth Edition, (DSM IV) published by the American Psychiatric Association or by the ICD (ICD-10: International Statistical Classification of Diseases and Related Health Problems (10th Revision) published by the World Health Organisation) or any other psychiatric classification system and a Hamilton score determines the severity of the depression. Hedlung, et al. The Hamilton Rating Scale for Depression, Journal of Operational Psychiatry (1979) 10 (2) 149-165. The patient population consisted of subjects aged 18-65 years (41±14). The severity of disease as measured by the Hamilton scale was 21-30. The healthy population consisted of individuals at the age of 18-50 years (35±9) and with a Hamilton scale of less than 3.

### Example 1

Apoptosis was measured in peripheral blood mononuclear cells (PBMC) by annexin V labeling (a membranal marker). The procedure was as follows:

1. PBMC were isolated on Ficoll gradient from 10 ml whole blood containing heparin.

2. The isolated PBMC were washed in phosphate buffered saline (PBS) and were resuspended in 2 ml RPMI (a liquid medium in which cells can live in) supplemented with 50 μg/ml gentamycin either with or without 10% Fetal Calf Serum (FCS).

3. The cell concentration was adjusted to 1.5×10⁶ cells/ml and the cells were cultured in a 24-well plate (1 ml/well) for 18 h at 37° C.

4. Following overnight incubation, cells from each well were transferred to FACS tube and were washed twice in 2 ml calcium buffer (containing 10 mM Hepes, 200 mM NaCl and 2 mM CaCl₂) at 500 g for 5 min. According to the manufacturers instructions, Annexin V labeling has to be performed in a calcium-containing buffer.

5. The cell pellet was resuspended in 400 μl of calcium buffer.

6. 10 μl of Annexin V-FITC (IQ products-IQP-120F, Groningen, The Netherland) were added to 100 μl cell suspension. The mixture was incubated for 20 min at 4° C.

7. 2 ml of calcium buffer were added to each tube and the cells were washed at 500 g for 5 min.

8. 10 μl of propidium iodide (PI, IQP, Groningen, The Netherland) were added and the cells were incubated for 10 min at 4° C. in the dark.

9. Cells were kept at 4° C. until they were analyzed by flow cytometer.

10. Flow cytometry was performed on a FACS Calibur machine (Becton Dickinson) by procedures well known in the art.

Annexin V binds to phosphatidylserine that is exposed to the outer leaflet of the membrane upon apoptosis, whereas, PI is a marker for cell death. By using the combinations of these two markers, apoptotic cells will be Annexin V positive and PI negative.

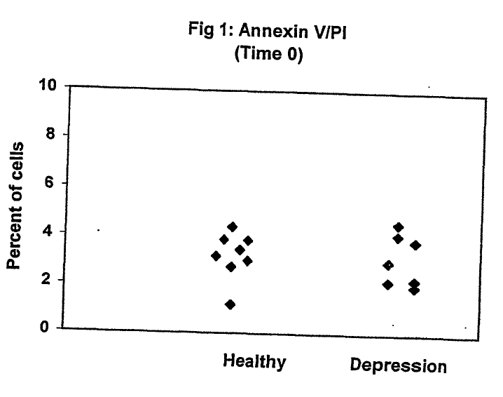

PBMC derived from healthy and depressed individuals were labeled by Annexin V/PI immediately after blood collection and PBMC isolation. A low level of apoptotic cells could be detected in both healthy and depressed individuals, with no difference in this level between the two populations (FIG. 1). These results are in contradiction to the results published in J. Immunol 1999 163(1):5334, where the authors describe an increase in apoptosis in the depressed population under these conditions.

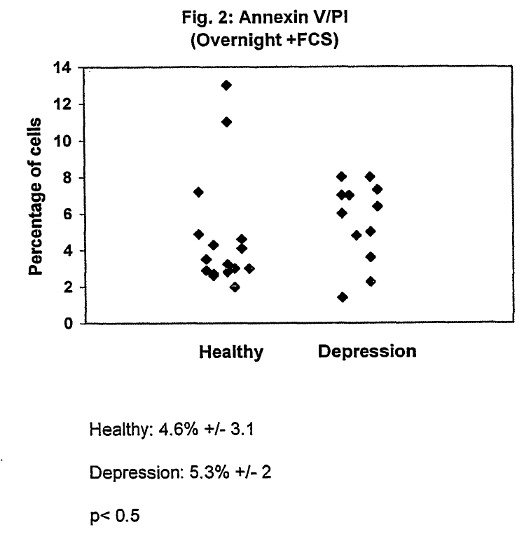

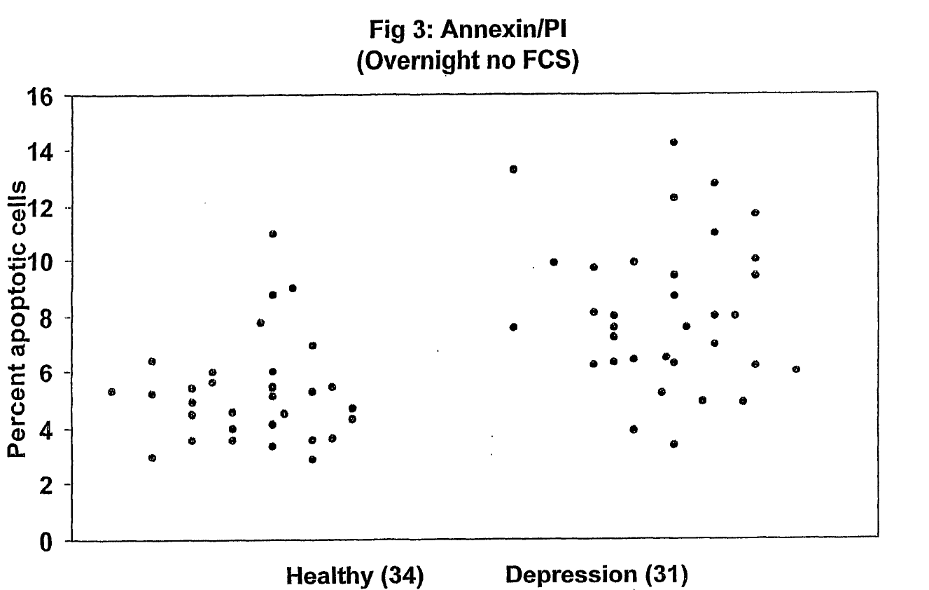

Apoptotic cells could be detected in PBMC samples only after they were cultured for 18 h at 37° C. PBMC samples that were cultured in the presence of 10% FCS did not differ in a statistically significant manner between healthy and depressed individuals (FIG. 2). However, PBMC samples, identical to those presented in FIG. 2, that were cultured in the absence of FCS showed a differential level of apoptotis (FIG. 3, Annexin V+/PI−) between healthy and depressed subjects. Cells derived from depressed individuals had a higher level of apoptotic cells (Annexin V+/PI−) as compared to the healthy population. This difference is highly significant statistically. It is already known that without continuous signaling by growth factors, hormones or cytokines as provided by FCS, cells undergo apoptosis. However, these results show that under these conditions PBMC derived from depressed individuals become much more susceptible to induction of apoptosis. The susceptibility to inducers of apoptosis can thus be used for the diagnosis of depression.

### Example 2

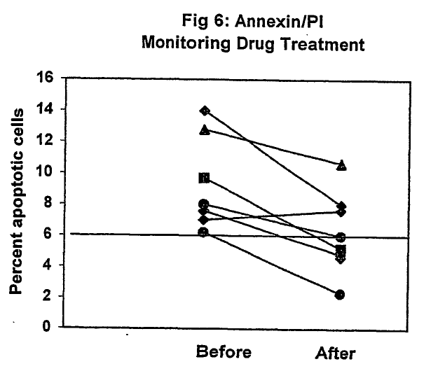

Patients were monitored using the procedures of Example 1. PBMC derived from blood samples of 7 depressed individuals were cultured overnight in the absence of FCS. Following incubation, cells were labeled by both Annexin V and PI and the level of apoptotic cells was measured. The same procedure was repeated on blood samples taken from the same individuals before treatment (“Before”) and after a successful treatment (“After”). The seven depressed individuals were treated with two different anti-depressant medications. The follow up of depressed patients (in whom the level of apoptotic cells was higher than normal), after drug treatment and clinical recovery or improvement, shows that the rate of apoptosis was significantly reduced after treatment as opposed to “pre treatment” (FIG. 6). The severity of disease as measured by the Hamilton scale was between 21-29 before treatment. All patients turned clinically healthy within a period of 21-30 days after the beginning of treatment. The ‘after treatment’ measurement was performed only after clinical improvement.

These results indicate that, under defined conditions, the extent of apoptosis and cell death in cell populations derived from depressed individuals is different than that observed for cell population derived from healthy individuals. The differential response of cells derived from depressed or healthy individuals to various stimuli, as measured by the percentage of apoptotic cells, in the treated samples, may serve as a diagnostic tool for depression. In particular, healthy and depressed populations can be distinguished by the response of cells derived from healthy or depressed individuals to different stimuli as measured by markers for different stages of apoptosis. Serum deprivation for 18 h generates more apoptotic cells in the population derived from depressed individuals as measured by Annexin VIPI staining.

### Example 3

Intracellular Proteins.

Pools of fresh PBMC's obtained from two groups of 10 healthy individuals and one group of 10 Major Depression patients were tested by Western blot for differential protein expression. Unlike Examples 1 and 2, these cells were not cultured prior to assay.

The proteins tested are known as apoptosis-related cellular proteins

1. Methodology

The PowerBlot western blotting and data analysis were as follows:

Western blotting—all steps are carried out at room temperature.

1) The gel is 16×16 cm, 5-15% gradient SDS-polyacrylamide, 1mm thick. A gradient system is used so a wide size range of proteins can be detected on one gel.

2) 400 ug of protein is loaded in one big well across the entire width of the gel. This translates into ˜15 ug per lane on a standard 25 well gel. The gel is run overnight at constant milliamps.

3) The gel is transferred to Immobilon-P nylon membrane (Millipore) for 1 hour at 1 amp. We use a wet electrophoretic transfer apparatus TE Series from Hoefer.

4) After transfer, the membrane is blocked for one hour with 5% milk.

5) Next, the membrane is clamped with a western blotting manifold that isolates 45 channels across the membrane. In each channel, a complex antibody cocktail is added and allowed to hybridize for one hour. The various proteins are detected and quantitated with separate monoclonal antibodies. Such monoclonal antibodies may be prepared by procedures well known in the art. Alternatively, the monoclonal antibodies are available from BD-Transduction Laboratories.

6) The blot is removed from the manifold, washed and hybridized for 30 minutes with secondary goat anti-mouse immunoglobulin conjugated to horse radish peroxidase (HRP). All antibodies are mouse monoclonals so only one secondary control is needed

7) The membrane is washed and developed with chemiluminescence. We use the SuperSignal West Pico from Pierce.

8) Chemiluminescent signals are captured using the Kodak Image Station CCD Camera.

9) Molecular Weight (MW) Standards—Standards are composed of an antibody cocktail added to lane 45 of PowerBlot gels.

The standard proteins and their molecular weights were as follows:

| p150Glued | 150 kD |
|---|---|
| Adaptin beta | 106 |
| STAT-3 | 92 |
| PTP1D | 72 |
| Mek-2 | 46 |
| RACK-1 | 36 |
| GRB-2 | 24 |
| Rap2 | 21 |

Data Analysis

Data analysis includes raw and normalized digital data from each blot with changes greater than 1.4 fold indicated. A description of characteristics of the analysis follow:

1) Quantity—total intensity of a defined spot.

2) Normalized Quantity—the raw quantity of a spot divided by the total intensity value of all pixels in an image multiplied by 1,000,000.

3) Standards Average—The average Quantity for normalization standards of verification blots. An actin antibody was used as the standard for normalization of verification blots.

4) Ratio—The Normalized Quantity for Treated bands expressed as a ratio of the Normalized Quantity for the corresponding Control bands. The Ratio is determined in order to express increases or decreases in protein expression.

5) Fold Change—Additionally, changes are expressed as Fold increase or decrease between Control and Treated Normalized Quantities.

Results

The Table below (Table 1) describes the fold change of amount of specified protein, between the Depressive patients and the healthy controls. A minus sign (“−”) before the number means that the specified protein was found to be expressed at a lower level in the Depressive patients. No sign means that the expression in the patients was found to be higher than in the controls. Table 1: Differential protein content of PBMC's obtained from Major Depression patients, as compared to healthy controls:

TABLE 1

| Protein | Fold change |
|---|---|
| Alpha-Catenin | −3 |
| VLA-2 (Very Late Antigen) | −6 |
| Apaf-1 (Apoptotic Protease-Activating Factor) | −40 |
| Nucleoporin p62 | 5 |
| Smad2 (Sma- and Mad-Related Proteins) | −3 |
| Hsp60 (Heat Shock Protein) | −2 |
| Integrin5 alpha | −2 |
| TRADD (TNFR1-Associated Death Domain) | 2 |
| Erk2 (Extracellular Signal Regulated Kinases) | −4 |
| JAK1 (Janus Kinase 1) | −2 |
| Ceruloplasmin | −2 |
| HAP1 (Huntigton-Associated Protein) | −2 |

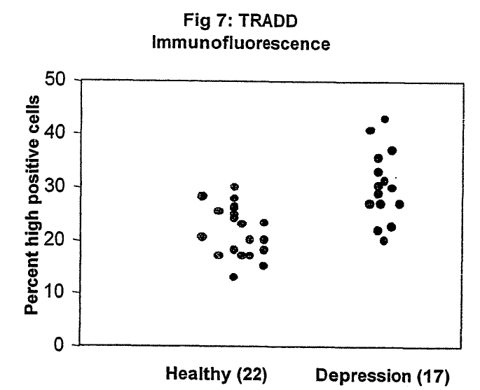

These same proteins can also be detected and measured in blood cells of individuals upon fixation with paraformaldehyde (4%) and permeabilization with saponin (0.1%) of their PBMC'S, followed by immunofluorescence and flow cytometry. Such a procedure is exemplified in FIG. 7, in which the intracellular protein TRADD has been measured in healthy and depressed individuals FIG. 7 shows that the percent of lymphocytes, highly positive for intracellular TRADD, is higher in the Depression group than in the healthy group. This difference is highly significant, statistically.

### Example 4

mRNA expression

The mRNA's obtained from fresh PBMC's, encoding for various apoptosis-related cellular proteins were analyzed and quantitated. Similarly to Example 3, these cells were not cultured prior to RNA analysis.

Methodology

GEArray—ASSAY PROTOCOL

1. Probe Synthesis

Total RNA is used as a template for biotinylated probe synthesis using the Following procedure:

Synthesis of cDNA probes with dNTP mix containing biotin-16-dUTP:

(1) Annealing

For each total RNA sample, combine the following into a sterile PCR tube:

| | | |
|---|---|---|
| Total RNA | 5-10 | μg |
| GEAprimer Mix | 2 | μl |

To each tube, add RNase-Free H₂O to a final volume of 20 μl.

Mix the contents well by gentle pipetting, centrifuge briefly. Place the mixture in a preheated heat block at 70° C. for 2 min. Cool to 42° C. and keep tube at 42° C. for 2 min before adding labeling mix.

(2) Prepare the labeling mix

For each total RNA sample, prepare 20 μl master labeling mix.

| | 1 sample | 2 samples |
|---|---|---|
| 5 X Normal-GEAlabeling | 8 μl | 16 μl |
| Biotin-16-dUTP (1 mM) | 4 μl | 8 μl |
| RNase inhibitor | 1 μl | 2 μl |
| MMLV Reverse Transcriptase (50 units/μl) | 2 μl | 4 μl |
| Rnase-free H2O | 5 μl | 10 μl |
| Final Volume | 20 μl | 40 μl |

(3) Labeling reaction

Pre-warm the labeling mix prepared in step (2) to 42° C. for 2 min. Transfer 20 μl of the labeling mix to each annealing reaction and mix the content well by gentle pipetting. Continue incubating at 42° C. for 120 min.

(4) Stop the labeling reaction by adding 5 μl of 10× Stop Solution (Buffer C).

(5) Denaturation of the reverse transcribed cDNA probe Add 5 μl of 10×denaturing Solution to the labeled cDNA probe (45 μl) and incubate at 68° C. for 20 min. Add 50 μl of 2×Neutralization Solution and continue the incubation at 68° C. for 10 min. The cDNA probe is ready to be added to the Hybridization Solution.

2. Hybridization

(1) Prewarm 15 ml of GEAhyb Hybridization Solution to 68° C. for each membrane.

(2) Heat-denature sheared salmon sperm DNA at 100° C. for 5 min, and chill quickly on ice. Add the heat-denatured salmon sperm DNA to the prewarmed GEAhyb Hybridization solution to a final concentration of 100 pg DNA/ml, and keep at 68° C. until use.

(3) Wet the GEArray membrane with deionized H₂O and place the membrane into a hybridization bottle or bag.

(4) Add 10 ml of Hybridization Solution prepared as described above. Pre-hybridize at 68° C. for 1 to 2 hours with continuous agitation at 5-10 rpm/min. Keep the remaining 5 ml of GEAhyb Hybridization Solution at 68° C. until step (6).

(5) Pour off the prehybridization solution and discard.

(6) Mix the denatured cDNA probe (100 μl) with the remaining 5 ml of GEAhyb hybridization Solution prepared in step (1). Hybridize overnight with continuous agitation at 68° C.

(7) Wash the membrane twice with 75 ml of pre-warmed wash Solution 1 (2×SSC, 1% SDS) for 20 min at 68° C. with agitation at 30-40 rpm/min.

(8) Wash the membrane twice with 75 ml pre-warmed wash solution 2 (0.1×SSC, 0.5% SDS) for 20 min at 68° C. with agitation at 30-40 rpm/min.

3. Chemiluminescence detection

(1) Blocking the GEArray membrane with GEAblocking solution:

After washing the GEArray membrane with washing solution 2, remove the membrane from the hybridization cylinder, and place it in a small clean tray.

Warm the GEAblocking solution bottle to 50° C. for 10 min, invent the bottle several times to mix, and cool the bottle to room temperature. Pipet 10 ml GEAblocking solution for each GEArray membrane. Incubate the GEArray membrane with GEAblocking solution at room temperature for 40 min with gentle shaking.

(2) Incubating with alkaline phosphatase-conjugated streptavidin:

Pour the GEAblocking solution from the tray into a new conical tube. Dilute the alkaline phosphatase-conjugated streptavidin (AP-streptavidin) 1:5,000 with the GEAblocking solution, and mix well.

Incubate the GEArray membrane with diluted AP-streptavidin at room temperature with gentle shaking for 40 min. (3) Washing the membrane:

Warm the 5× washing buffer F to 37° C. in a water bath. Dilute 5X washing buffer with dH₂O to make 1× washing buffer (20 ml 5X buffer F+80 ml H₂O).

Wash the membrane with 10 ml of 1× washing buffer for 5 min with gentle shaking. Repeat the washing three times.

After finishing the final wash, rinse the membrane twice with 10 ml 1× AP-assay buffer.

(4) Detecting the GEArray by chemiluminescence:

Drain AP-assay buffer completely. Incubate the GEArray membrane with 8 ml CDP-Star chemiluminescent substrate for 2 min with gently shaking.

Blot the membrane on a piece of filter paper to remove excess CDP-Star. Place the membrane in a hybridization bag smooth out bubbles and measure the chemiluminescence intensity by a chemiluminescence reader.

(5) Determination of the relative abundance of transcripts.

Each GEArray membrane is spotted with a negative control of pUC18 as well as two positive control genes, β-actin and GAPDH. The relative abundance of a particular transcript can be estimated by comparing its signal intensity to the signal derived from β-actin and/or GAPDH.

Results

The following Table (Table 2), describes the relative mRNA amounts, encoding the specified proteins, in PBMC's obtained from Major Depression patients or healthy controls. Patients and Controls are designated by numbers.

TABLE 2

| | | | | | |
|---|---|---|---|---|---|
| Healthy# | Level | SD | Depression# | Level | SD |

Bak

| | | | | | |
|---|---|---|---|---|---|
| 553 | 504 | | 591 | 197 | |
| 607 | 323 | | 649 | 74 | |
| 578 | 245 | | 653 | 192 | |
| 671 | 284 | | 652 | 125 | |
| 659 | 303 | | 658 | 98 | |
| 1519 | 402 | | 1521 | 242 | |
| Mean | 344 | 94 | Mean | 155 | 65 |

Caspase 1

| | | | | | |
|---|---|---|---|---|---|
| 1414 | 26 | | 1457 | 206 | |
| 1458 | 130 | | 1548 | 307 | |
| 587 | 328 | | 602 | 350 | |
| 599 | 182 | | 604 | 237 | |
| 603 | 161 | | 629 | 185 | |
| 676 | 149 | | 648 | 372 | |
| Mean | 163 | 98 | Mean | 276 | 78 |

Birc 3

| | | | | | |
|---|---|---|---|---|---|
| 1414 | 0 | | 1457 | 221 | |
| 1458 | 138 | | 1548 | 342 | |
| 587 | 410 | | 602 | 204 | |
| 599 | 172 | | 604 | 206 | |
| 603 | 112 | | 629 | 140 | |
| 676 | 74 | | 648 | 262 | |
| Mean | 151 | 140 | Mean | 229 | 68 |

Hus 1

| | | | | | |
|---|---|---|---|---|---|
| 1414 | 0 | | 1457 | 136 | |
| 1458 | 15 | | 1548 | 46 | |
| 587 | 94 | | 602 | 28 | |
| 599 | 39 | | 604 | 72 | |
| 603 | 30 | | 629 | 98 | |
| 676 | 10 | | 648 | 97 | |
| Mean | 31 | 34 | Mean | 80 | 39 |

Bcl 2

| | | | | | |
|---|---|---|---|---|---|
| 1414 | 174 | | 1457 | 335 | |
| 1458 | 71 | | 1548 | 584 | |
| 587 | 160 | | 602 | 290 | |
| 599 | 186 | | 604 | 234 | |
| 603 | 174 | | 629 | 0 | |
| 676 | 238 | | 648 | 317 | |
| Mean | 167 | 54 | Mean | 293 | 188 |

Birc 6

| | | | | | |
|---|---|---|---|---|---|
| 1414 | 378 | | 1457 | 309 | |
| 1458 | 417 | | 1548 | 127 | |
| 587 | 431 | | 602 | 174 | |
| 599 | 416 | | 604 | 289 | |
| 603 | 233 | | 629 | 265 | |
| 676 | 319 | | 648 | 298 | |
| Mean | 366 | 77 | Mean | 244 | 75 |

We claim:

1. A method of diagnosing a subject having depression comprising:
a) obtaining a sample of cells of the subject and b) determining the level of one or more apoptotic related proteins in said
cells thereby diagnosing the subject having depression.

2. The method of claim 1 wherein said level of apoptotic related proteins is determined by western blot analysis, chip, protein chip, immunofluorescence, flow cytometry or enzyme linked immunosorbent assay techniques.

3. The method of claim 1 wherein said proteins are selected from the group consisting of: Alpha-Catenin; Very Late Antigen; Apoptotic Protease-Activating Factor; Nucleoporin p62; Sma- and Mad-Related Proteins; Heat Shock Protein 60; Integrin 5 alpha protein; Tumor Necrosis Factor-1 Associated Death Domain; Extracellular Signal Regulated Kinases; Janus Kinase 1; Huntington-Associated Protein and Ceruloplasmin.

4. A method of diagnosing a subject having depression comprising
a) obtaining a sample of cells of the subject and
b) determining the mRNA levels of one or more depression-related genes in said cells thereby diagnosing the subject having depression.

5. The method of claim 4 wherein said mRNA levels are determined by northern or PCR analysis.

6. The method of claim 4 wherein said mRNA levels are determined by chip analysis.

7. The method of claim 4 wherein the depression-related gene is selected from the group consisting of caspase 1, 5, 8, bak, Birc 3, Birc 6, Hus 1 and Bcl2.

8. A method of monitoring the progression of a depression disorder of a subject, comprising:
a) obtaining a sample of cells of the subject a more than one time point and
b) determining the level of one or more apoptotic related proteins over time in said cells thereby monitoring the progression of a depression disorder of said subject.

9. The method of claim 8 wherein said level of apoptotic related proteins is determined by western blot analysis, chip, protein chip, immunofluorescence, flow cytometry or enzyme linked immunosorbent assay techniques.

10. The method of claim 8 wherein said proteins are selected from the group consisting of: Alpha-Catenin; Very Late Antigen; Apoptotic Protease-Activating Factor; Nucleoporin p62; Sma- and Mad-Related Proteins; Heat Shock Protein 60; Integrin 5 alpha protein; Tumor Necrosis Factor-1 Associated Death Domain; Extracellular Signal Regulated Kinases; Janus Kinase 1; Huntington-Associated Protein and Ceruloplasmin.

11. A method of monitoring the progression of a depressive disorder of a subject, comprising:
a) obtaining a sample of cells of the subject at more than one time point and
b) determining the mRNA levels of one or more depression-related genes over time in said cells thereby monitoring the progression of a depression disorder of said subject.

12. The method of claim 11 wherein said mRNA levels are determined by northern or PCR analysis.

13. The method of claim 11 wherein said mRNA levels are determined by chip analysis.

14. The method of claim 11 wherein the depression-related gene is selected from the group consisting of caspase 1, 5, 8, bak, Birc 3, Birc 6, Hus 1 and Bcl2.

15. The method of claims 8 -14 wherein said subject is diagnosed as having depression by well recognized clinical set of criteria as outlined by DSM (Diagnostic and Statistical Manual of Mental Disorders, Fourth Edition, (DSM IV) published by the American Psychiatric Association) or the ICD (ICD-10: International Statistical Classification of Diseases and Related Health Problems (10th Revision) or any other psychiatric classification system.

16 A method of diagnosing a subject having depression comprising:
a) obtaining a sample of cells of the subject;
b) incubating the sample in a serum deprived cell culture medium; and
c) determining the percentage of apoptotic cells in said sample thereby diagnosing the subject having depression.

17. The method of claim 16 wherein said percentage of apoptotic cells is determined by annexin V labeling.

18. A method of monitoring the progression of a depressive disorder of a subject, comprising:
a) obtaining a first sample of cells of the subject;
b) incubating the first sample in a serum deprived medium;
c) contacting the sample with a binding molecule, wherein the binding molecule is capable of specifically binding to an apoptotic cellular marker, so as to form a complex between the binding molecule and the marker,
d) determining the percentage of cells that possess the binding molecule in the first sample;
e) obtaining a second sample of cells of the subject;
f) incubating the second sample in a serum deprived medium;
g) contacting the second sample with a binding molecule, wherein the binding molecule is capable of specifically binding to an apoptotic cellular marker, so as to form a complex between the binding molecule and the marker;
h) determining the percentage of cells that possess the binding molecule in the second sample; and
i) comparing percentage of cells obtained in step d) with the level obtained in step h), thereby monitoring the progression of the subject having the depressive disorder.

19. The method of claim 18 wherein said binding molecule is annexin V.

20. A method of monitoring the therapeutic efficacy of a treatment in a subject having a depressive disorder comprising:
a) obtaining a sample of cells of the subject before and after treatment of said subject and
b) determining the level of one or more apoptotic related proteins over time in said cells thereby monitoring the progression of a depression disorder of said subject.

21. The method of claim 20 wherein said level of apoptotic related proteins is determined by western blot analysis, chip, protein chip, immunofluorescence, flow cytometry or enzyme linked immunosorbent assay techniques.

22. The method of claim 20 wherein said proteins are selected from the group consisting of: Alpha-Catenin; Very Late Antigen; Apoptotic Protease-Activating Factor; Nucleoporin p62; Sma- and Mad-Related Proteins; Heat Shock Protein 60; Integrin 5 alpha protein; Tumor Necrosis Factor-1 Associated Death Domain; Extracellular Signal Regulated Kinases; Janus Kinase 1; Huntington-Associated Protein and Ceruloplasmin.

23. A method of monitoring the progression of a depression disorder of a subject, comprising:
a) obtaining a sample of cells of the subject before and after treatment of said subject and
b) determining the mRNA levels of one or more depression-related genes in said cells over time thereby monitoring the progression of a depression disorder of said subject.

24. The method of claim 23 wherein said mRNA levels are determined by northern analysis, PCR or chip analysis.

25. The method of claim 23 wherein the depression-related gene is selected from the group consisting of caspase 1, 5, 8, bak, Birc 3, Birc 6, Hus 1 and Bcl2.

26. A method of monitoring the therapeutic efficacy of a treatment in a subject having a depressive disorder comprising:
a) obtaining a first sample of cells of the subject;
b) incubating the first sample in a serum deprived medium
c) contacting the first sample with a binding molecule, wherein the binding molecule is capable of specifically binding to an apoptotic cellular marker, so as to form a complex between the binding molecule and the marker;
d) determining the percentage of cells that possess the binding molecule in the first sample;
e) treating the subject with an antidepressant mood stabilizing, or other treatment or a combination of treatments; f) obtaining a second sample of cells of the subject;
g) incubating the second sample in the presence of an apoptotic inducer;
h) contacting the second sample with a binding molecule, wherein the binding molecule is capable of specifically binding to an apoptotic cellular marker, so as to form a complex between the binding molecule and the marker;
i) determining the percentage of cells that possess the binding molecule in the second sample; and
j) determining the therapeutic efficacy of the treatment based on the percentage of labeled cells, thereby monitoring the therapeutic efficacy of treatment in the subject having depression.

27. The method of claim 26 wherein said binding molecule is annexin V.

28. The method of claims 18-27 wherein said subject is diagnosed as having depression by well recognized clinical set of criteria as outlined by DSM (Diagnostic and Statistical Manual of Mental Disorders, Fourth Edition, (DSM IV) published by the American Psychiatric Association) or the ICD (ICD-10: International Statistical Classification of Diseases and Related Health Problems (10th Revision) or any other psychiatric classification system

29. A kit for detecting a depressive disorder in a patient, comprising: one or more apoptotic cell detection components.

30. The kit of claim 29 wherein said apoptotic cell detection component is annexin V.

31. A kit for detecting a depressive disorder in a patient, comprising: one or more apoptosis-related protein detection components.

32. The kit of claim 31 wherein said detection component further comprises antibodies to one or more proteins selected from the group consisting of: Alpha-Catenin; Very Late Antigen; Apoptotic Protease-Activating Factor; Nucleoporin p62; Sma- and Mad-Related Proteins; Heat Shock Protein 60; Integrin 5 alpha protein; Tumor Necrosis Factor-1 Associated Death Domain; Extracellular Signal Regulated Kinases; Janus Kinase 1; Huntington-Associated Protein and Ceruloplasmin.

33. A kit for detecting a depressive disorder in a patient, comprising: one or more depression-related gene detection components.

34. The kit of claim 33 wherein the depression-related gene detection component is an mRNA probe for an mRNA selected from the group consisting of caspase 1, 5, 8, bak, Birc 3, Birc 6, Hus 1 and Bcl2 mRNAs.

35. A method of treating a patient with a depressive disorder comprising administering an anti-apoptotic drug to said patient.

36. The method of claim 35 wherein said subject is diagnosed as having depression by well recognized clinical set of criteria as outlined by DSM (Diagnostic and Statistical Manual of Mental Disorders, Fourth Edition, (DSM IV) published by the American Psychiatric Association) or the ICD (ICD-10: International Statistical Classification of Diseases and Related Health Problems (10th Revision) or any other psychiatric classification system
